(19) United States
(12) Patent Application Publication
(10) Pub. No.: US 2022/0403426 A1
(43) Pub. Date: Dec. 22, 2022

(54) METHOD OF STORING A BIOCATALYST

(71) Applicant: BASF SE, Ludwigshafen am Rhein (DE)

(72) Inventors: Diego GHISLIERI, Ludwigshafen am Rhein (DE); Peter OEDMAN, Ludwigshafen am Rhein (DE); Tobias Joachim ZIMMERMANN, Ludwigshafen am Rhein (DE)

(21) Appl. No.: 17/774,463

(22) PCT Filed: Nov. 4, 2020
(86) PCT No.: PCT/EP2020/080887

§ 371 Date: May 4, 2022

(30) Foreign Application Priority Data
Nov. 5, 2019 (EP) 19207071.2

(51) Int. Cl. C12P 13/02; C12N 9/88; C12N 1/20; C12N 1/04

(52) U.S. Cl. CPC C12P 13/02; C12N 9/88; C12N 1/205; C12N 1/04

(57) ABSTRACT

The present invention relates to a method of storing a biocatalyst, which biocatalyst is capable of converting acrylonitrile to acrylamide, comprising the steps: (a) providing an aqueous suspension comprising the biocatalyst, which is capable of converting acrylonitrile to acrylamide, which aqueous suspension is an aqueous fermentation broth; (b) sequentially in either order or simultaneously (b) concentrating the aqueous suspension comprising the biocatalyst to a concentration of at least 3% (w/w); and (b2) reducing the temperature of the aqueous suspension comprising the biocatalyst to a temperature of below 8° C., thereby forming a concentrated aqueous suspension; and (c) maintaining the concentrated aqueous suspension of step (b) at a temperature of below 8° C. The invention also relates to a biocatalyst composition obtainable by this method and also the use of the biocatalyst composition in a process of preparing (meth-) acrylamide aqueous solution from (meth-) acrylonitrile. The invention further relates to a method for producing an aqueous (meth-) acrylamide solution comprising the aforementioned storage method and also to a method for producing polyacrylamide comprising the aforementioned storage method.

### BACKGROUND OF THE INVENTION

### Field of the Invention

The present invention relates to methods for storing biocatalysts that are capable of converting acrylonitrile to acrylamide. The present invention also relates to biocatalyst compositions obtainable by the method of storage and also relates to uses of such biocatalysts that have undergone the inventive method of storing for preparing aqueous solutions of (meth-) acrylamide. Further, the present invention concerns a process for preparing an aqueous (meth-) acrylamide solutions and polyacrylamides employing biocatalyst compositions comprising the method of storage of the biocatalysts.

### Description of the Prior Art

Polyacrylamides are widely used as flocculants for a variety of industries including the mining industry. Other common uses of polyacrylamides include additives for enhanced oil recovery and drift reduction additives for soil treatment in agricultural applications. The raw material for polyacrylamide is typically its monomer acrylamide. In principle, there are two different methods of producing acrylamide on an industrial scale: chemical synthesis and biological synthesis, wherein the biological synthesis methods are more and more on the rise due to mild reaction conditions and inherent process safety. Due to the milder reaction conditions, the absence of copper catalyst and the quantitative conversion of the nitrile, expensive downstream processing steps such as distillation or ion exchange can be avoided in the biological synthesis, thus resulting in cheaper plants with drastically reduced plant footprint.

Both synthesis methods use acrylonitrile as starting substance. While the chemical synthesis method uses copper catalyst (e.g. U.S. Pat. Nos. 4,048,226, 3,597,481), the biological synthesis method (also known as bio-based method) employs biocatalysts to hydrate (i.e. to convert) acrylonitrile in order to obtain acrylamide. Generally, such biocatalysts are microorganisms which are capable of producing (i.e. which encode) the enzyme nitrile hydratase (IUBMB nomenclature as of Sep. 30, 2014: EC 4.2.1.84; CAS-No. 2391-37-5; also referred to as, e.g., NHase). Nitrile hydratase producing microorganisms are largely distributed in the environment and comprise, inter alia, representatives of the species Rhodococcus rhodochrous, Rhodococcus pyridinovorans, Rhodococcus erythropolis, Rhodococcus equi, Rhodococcus ruber, Rhodococcus opacus, Aspergillus niger, Acidovorax avenae, Acidovorax facilis, Agrobacterium tumefaciens, Agrobacterium radiobacter, Bacillus subtilis, Bacillus pallidus, Bacillus smithii, Bacillus sp BR449, Bradyrhizobium oligotrophicum, Bradyrhizobium diazoefficiens, Bradyrhizobium japonicum, Burkholderia cenocepacia, Burkholderia gladioll, Klebsiella oxytoca, Klebsiella pneumonia, 35 Klebsiella variicola, Mesorhizobium ciceri, Mesorhizobium opportunistum, Mesorhizobium sp F28, Moraxella, Pantoea endophytica, Pantoea agglomerans, Pseudomonas chlororaphis, Pseudomonas putida, Rhizobium, Rhodopseudomonas palustris, Serratia liquefaciens, Serratia marcescens, Amycolatopsis, Arthrobacter, Brevibacterium sp CH1, Brevibacterium sp CH2, Brevibacterium sp R312, Brevibacterium imperiale, Corynebacternum nitrilophilus, Corynebacterium pseudodiphteriticum, Corynebacterium glutamicum, Corynebacterium hoffmanii, Microbacterium imperiale, Microbacterium smegmatis, Micrococcus luteus, Nocardia globerula, Nocardia rhodochrous, Pseudonocardia thermophila, Trichoderma, Myrothecium verrucaria, Aureobasidium pullulans, Candida famata, Candida guilliermondii, Candida tropicalis, Cryptococcus flavus, Cryptococcus sp UFMG-Y28, Debaryomyces hanseii, Geotrichum candidum, Geotrichum sp JR1, Hanseniaspora, Kluyveromyces thermotolerans, Pichia kluyveri, Rhodotorula glutinis, Comomonas testosteroni, Pyrococcus abyssi, Pyrococcus furiosus, and Pyrococcus horikoshi. (see, e.g., Prasad, Biotechnology Advances (2010), 28(6): 725-741; FR2835531). The enzyme nitrile hydratase is either iron- or cobalt-dependent (i.e. it possesses either an iron or a cobalt atom coordinated in its activity center) which is particularly characterized by its ability to catalyze conversion of acrylonitrile to obtain acrylamide by hydrating acrylonitrile (Kobayashi, Nature Biotechnology (1998), 16: 733-736).

EP 2716765 A1 describes a method for producing acrylamide comprising: supplying a raw material water to a reactor, supplying acrylonitrile to the reactor, and hydrating acrylonitrile using a biocatalyst, wherein a temperature of the raw material water in the supplying step of the raw material water to the reactor is equal to or more than the freezing point of the raw material water and lower than the reaction temperature by 5° C. or more. Example 1 describes preparing a biocatalyst employing Rhodococcus rhodochrous J1 having nitrile hydratase activity by aerobically culturing in a medium containing 2% glucose, 1% urea, 0.5% peptone, 0.3% yeast extract and 0.05% cobalt chloride (pH 7.0) at 30° C. The obtained culture was subjected to harvest and a washing step in order to prepare a bacterial cell suspension as a biocatalyst.

Biocatalysts are, however, prone to losing activity if subjected to harsh conditions, such as excessive temperature or starvation etc. Nevertheless, it is often necessary to prepare such microorganisms for storage, for instance to retain the microorganism for later use or for transporting to a different location for subsequent use. A commonly used technique for biocatalyst storage is to first subject it to a spray drying process and to then put the so formed dried biocatalyst into a storage container. It has been found that the biocatalyst can lose activity during the spray drying process and also during storage. Inevitably a biocatalyst with reduced activity is less efficient at converting acrylonitrile to acrylamide.

It would therefore be desirable to provide a more effective method of storing biocatalysts that are capable of converting acrylonitrile to acrylamide.

This objective technical problem has been overcome by the present invention as defined in the claims and as described and exemplified herein below.

### SUMMARY OF THE INVENTION

The present invention provides a method of storing a biocatalyst, which biocatalyst is capable of converting acrylonitrile to acrylamide, comprising the steps:

(a) providing an aqueous suspension comprising the biocatalyst, which is capable of converting acrylonitrile to acrylamide, which aqueous suspension is an aqueous fermentation broth;

(b) sequentially in either order or simultaneously
(b1) concentrating the aqueous suspension comprising the biocatalyst to a concentration of at least 3% (w/w); an
(b2) reducing the temperature of the aqueous suspension comprising the biocatalyst to a temperature of below 8° C.,

thereby forming a concentrated aqueous suspension; and

(c) maintaining the concentrated aqueous suspension of step (b) at a temperature of below 8° C.

The inventors have found that this method of storing the biocatalyst provides improved retention of activity and improve stability. Further, there is fermentation capacity required. The inventive method provides an effective way of storing biocatalysts which negates any requirement for spray drying. As a result of these advantages the storage method means that the biocatalyst can be used much more cost effectively.

### DESCRIPTION OF DRAWINGS

FIG. 1 illustrates the results in terms of initial specific rate (mol/h*gcat) and dosing time (hours) over time for Experiment Series I in which the stored biocatalyst has a solids of 23% (w/w) and a storage temperature of −20° C.

FIG. 2 illustrates the results in terms of initial specific rate (mol/h*gcat) and dosing time (hours) over time for Experiment Series II in which the stored biocatalyst has a solids of 16% (w/w) and a storage temperature of −20° C.

FIG. 3 illustrates the results in terms of initial specific rate (mol/h*gcat) and dosing time (hours) over time for Experiment Series III in which the stored biocatalyst has a solids of 16% (w/w) and a storage temperature of 4° C.

FIG. 4 illustrates the results in terms of initial specific rate (mol/h*gcat) and dosing time (hours) over time for the Control Experiment Series employing spray dried biocatalyst.

The unit gcat relates to the total dry mass of biocatalyst in grams.

### DETAILED DESCRIPTION

The aqueous biocatalyst suspension in step (a) is an aqueous fermentation broth. Desirably the aqueous fermentation broth includes a microorganism that has been cultivated for the expression of nitrile hydratase that would catalyse the bioconversion of acrylonitrile (ACN) to acrylamide (ACM). This may, for instance, be a fermentation broth that has been used in a process converting acrylonitrile to acrylamide. Suitably, the aqueous suspension comprising the biocatalyst is subjected to the inventive method shortly after the completion of the conversion of acrylonitrile to acrylamide. Preferably, the concentrating of the fermentation broth in step (b) of the inventive method is started within 5 hours, preferably 2 hours, more preferably 1 hour, most preferably 13 minutes after completion of the conversion of acrylonitrile to acrylamide. In a more preferred embodiment, the concentration step is started within 20 minutes after completion of the conversion of acrylonitrile to acrylamide. In an even more preferred embodiment, the concentration step is started within 10 minutes after completion of the conversion of acrylonitrile to acrylamide.

By biocatalyst we mean the total solids content including the microorganism or components of the microorganism, such as cellular material, enzymes or any other biological material; solids derived from the buffer, salts or any other additives present in suspension.

In accordance with the present invention the concentration of the aqueous biocatalyst suspension in step (b1) is increased to at least 3% (w/w). Suitably the concentration of the biocatalyst may be increased in this step to a concentration of at least 5% (w/w), desirably at least 8% (w/w) and specifically to at least 10% (w/w). Preferably, the concentration of the-biocatalyst may be increased to at least 15% (w/w) and more preferably to at least 20% (w/w). Examples of suitable concentrations which the biocatalyst may be increased to in step (b1) may lie in the ranges of from 3% to 60% (w/w) 5% to 50% (w/w), from 8% to 45% (w/w), from 8% to 40% (w/w), from 10% to 35% (w/w), from 10% to 30% (w/w), from 15% to 30% (w/w) or from 20% to 30% (w/w).

The concentrating of the biocatalyst in step (b1) to the desired concentration may be carried out in a single concentrating step. However, in some cases it may be desirable to separate the biocatalyst solids from the aqueous medium of the aqueous biocatalyst followed by resuspension in an aqueous medium at the desired concentration e.g. a concentration of at least 3% (w/w).

The concentrating of the aqueous biocatalyst suspension in step (b1) may employ any suitable concentrating means described in the patents and literature. Suitable means for achieving a higher concentration may for instance employ centrifugation or filtration or a combination of these techniques. Filtration may be achieved by any of the known filtration techniques described in the literature and patents, including for instance ultrafiltration. Filtration may be achieved by crossflow filtration or by the use of dead-end filters or using standard filter presses etc. Nevertheless, centrifugation techniques for increasing the concentration have been found to be more convenient. A preferred form of centrifugation for this concentrating step employs disk stack separation.

It may be desirable to concentrate the biocatalyst suspension in step (b1) in more than one stage. Thus, it may be desirable to concentrate the biocatalyst suspension to a concentration of from 3 to 16% solids content (w/w) in a first stage, for instance using disk stack separation. Suitably, the concentrating stage may achieve a concentration of from 10 to 16% solids content (w/w) and desirably from 12 to 16% (w/w), particularly 14 to 16% (w/w). For many applications it may not be necessary to concentrate the biocatalyst suspension any further. In some situations it may be desirable to achieve a higher concentration and this may be achieved by further concentrating this already concentrated biocatalyst suspension further, typically to achieve a concentration which is beyond 16% (w/w). Desirably this may be achieved for instance using centrifugation or filtration techniques.

In some embodiments of the present invention, the concentrating of the aqueous biocatalyst suspension is performed by disk stack separator. Any flow rate chosen will depend upon the size of the disk stack separator. On a relatively small plant setup such a flow rate may be as low as 500 L/h, for instance from 500 to 1000 L/h. However, typical production scale plants would tend to require a much higher flow rate, for instance from 2500 to 7000 L/h, such as from 3500 to 4000 L/h. Nevertheless, the optimal flow rate will always depend on the particular size of the disk stack separator used in the process and this can easily be established by a person skilled in the art.

Specific settling values are preferred in the context of the present invention, in particular in the concentration step of means, such as product or composition, methods or users of the present invention. The physical meaning of the specific settling area is the equivalent settling area of the centrifuge divided by the liquid feed flow rate to the centrifuge. Accordingly, it is a preferred embodiment of the means, methods and uses of the present invention that the concentration of the biocatalyst be performed such that the specific settling area is 118.0 m²h/l or less, preferably 60 m²h/l or less, more preferably 40 m²h/l or less. The unit m²h/l relates to metres squared hours per litre. In general, the lower the value of specific settling area the greater would be the separation.

It may be desirable to adjust the pH during or after the step (b). Typically, it may be desirable for the concentrated aqueous biocatalyst suspension to have a relatively neutral pH. Suitably, this may be, for instance, a pH of from 5.5 to 8.5, often from 6 to 8, normally from 6.5 to 7.5, particularly around pH 7. Suitably, the pH may be adjusted to an acidic pH, for instance from pH 4.5 to below 7.0, for instance from pH 5 to 6.5, such as from pH 5 to 6. PH adjustment of the concentrated aqueous biocatalyst suspension may be achieved by adding acidic or basic substances, for instance aqueous solutions of ammonia or phosphoric acid, respectively, depending upon the desired pH for the concentrated biocatalyst suspension.

In step (b2) of the inventive method the temperature of the aqueous biocatalyst suspension is reduced to a temperature of below 8° C. Preferably, the temperature in step (b2) is reduced to a temperature of up to 5° C. Further, the temperature may be reduced to up to 4° C. Typically, the temperature may be a temperature as low as −50° C. or lower. There is no minimum temperature below which the invention is not applicable. However, for practical purposes the temperature is usually reduced to a temperature of no lower than −30° C. Desirably, in step (b2) the concentrated suspension may be reduced to a temperature that lies within any of the ranges of from −50° C. to 8° C., from −30° C. to 8° C., typically from −25° C. to 5° C., often from −25° C. to 4° C., such as from −20° C. to 4° C., from −20° C. to 3° C., desirably from −20° C. to 2° C., for instance from −20° C. to 1° C. and suitably from −20° C. to 0° C. In one aspect of the invention the aqueous biocatalyst suspension temperature is reduced to a temperature at which the aqueous biocatalyst suspension is not frozen, for instance from 0° C. to 4° C.

In step (b) the concentration step (b1) and the temperature reduction step (b2) may be conducted in either order or simultaneously. Thus, where steps (b1) and (b2) are conducted sequentially, in accordance with the invention the concentration step (b1) may be carried out first followed by the temperature reduction step (b2); or also in accordance with the invention the temperature reduction step (b2) may be conducted first followed by the concentration step (b1). Where the temperature is to be reduced to below freezing the temperature reduction step (b2) if conducted first should be initially to a temperature of below 8° C. but at which the aqueous biocatalyst suspension does not freeze and then the concentration step (b1) should be completed prior to continuing the temperature reduction to the desired temperature below freezing. Where the temperature is to be reduced to above the freezing temperature step (b2) may be completed before initiating the concentration step (b1). It may also be desirable to combine the concentration step (b1) and temperature reduction step (b2) simultaneously. Nevertheless, when the temperature reduction in step (b2) is to a temperature below freezing, as above, the temperature reduction should only be initially to a temperature at which the aqueous biocatalyst suspension does not freeze before completing the concentration step (b1) and thereafter further temperature reduction to the desired temperature below freezing may be carried out.

It is, however, preferable that the concentration step (b1) is carried out before the temperature reduction step (b2).

According to step (c) of the inventive method the concentrated aqueous biocatalyst suspension of step (b) should be maintained at a temperature of below 8° C. By maintaining the temperature we mean that the concentrated aqueous biocatalyst suspension of step (b) is maintained at a temperature within the range of below 8° C. for the duration of the desired period of time of storage. This may, for instance, be for the duration of several hours or several days or even several months. Desirably, in step (c) the concentrated aqueous biocatalyst suspension should be maintained at a temperature of below 8° C. for at least 1 day, preferably at least 1 week, typically at least 2 weeks, more preferably at least 1 month and in particular at least 3 months. It may be required that the concentrated aqueous biocatalyst suspension be maintained in step (c) for longer. Still, for instance periods of up to 6 months or more, up to 9 months or more or up to 1 year or more, such as periods of from 1 day to 1.5 years, from 2 weeks to 1 year, from 1 month to 9 months or from 3 months to 6 months.

Suitably, in step (c) the temperature may be maintained at a substantially constant temperature and typically at a temperature obtained in step (b). Preferably, the temperature in step (c) is maintained at a temperature of up to 5° C. Typically, the temperature may be significantly lower than this, for instance −50° C. or lower. There is no minimum temperature below which the invention is not applicable. However, for practical purposes the temperature is usually maintained an a temperature of no lower than −30° C. Desirably, in step (c) the concentrated biocatalyst suspension may be maintained at a temperature that lies within any of the ranges of from −50° C. to 8° C., from −30° C. to 8° C., typically from −25° C. to 5° C., often from −25° C. to 4° C., such as from −20° C. to 4° C., from −20° C. to 3° C., desirably from −20° C. to 2° C., for instance from −20° C. to 1° C. and suitably from −20° C. to 0° C. In one aspect of the invention the concentrated biocatalyst suspension is maintained at a temperature at which the suspension is not frozen, for instance from 0° C. to 4° C. There may be some temperature variance during the period of maintaining the temperature provided that the maintenance temperature is within the specified range of the inventive method. Typically, the temperature during the maintenance step (c) will tend not to vary by more than 10° C., preferably by not more than 5° C.

In some embodiments of the present invention, the biocatalyst is a biocatalyst having nitrile hydratase activity. According to the present invention, the biocatalyst having nitrile hydratase activity may be one selected from the group consisting of microorganisms belonging to Rhodococcus, Aspergillus, Acidovorax, Agrobacterium, Bacillus, Bradyrhizobium, Burkholderia, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Escherichia, Geobacillus, Comomonas, and Pyrococcus, and transformed microbial cells in which a nitrile hydratase gene is introduced. In preferred embodiments of the invention the biocatalyst is selected from the group consisting of Rhodococcus, pseudomonas, Escherichia and Geobacillus.

In some embodiments the biocatalyst is Rhodococcus erythropolis. In some embodiments the biocatalyst is Rhodococcus equi. In some embodiments the biocatalyst is Rhodococcus ruber In some embodiments the biocatalyst is Rhodococcus opacus. In some embodiments the biocatalyst is Rhodococcus pyridinovorans. In some embodiments the biocatalyst is Aspergillus niger. In some embodiments the biocatalyst is Acidovorax avenae. In some embodiments the biocatalyst is Acidovorax facilis. In some embodiments the biocatalyst is Agrobacterium tumefaciens. In some embodiments the biocatalyst is Agrobacterium radiobacter In some embodiments the biocatalyst is Bacillus subtilis. In some embodiments the biocatalyst is Bacillus pallidus. In some embodiments the biocatalyst is Bacillus smithii. In some embodiments the biocatalyst is Bacillus sp BR449. In some embodiments the biocatalyst is Bradyrhizobium oligotrophicum. In some embodiments the biocatalyst is Bradyrhizobium diazoefficiens. In some embodiments the biocatalyst is Bradyrhizobium japonicum. In some embodiments the biocatalyst is Burkholderia cenocepacia. In some embodiments the biocatalyst is Burkholderia gladioli. In some embodiments the biocatalyst is Klebsiella oxytoca. In some embodiments the biocatalyst is Klebsiella pneumonia. In some embodiments the biocatalyst is Klebsiella variicola. In some embodiments the biocatalyst is Mesorhizobium ciceri. In some embodiments the biocatalyst is Mesorhizobium opportunistum. In some embodiments the biocatalyst is Mesorhizobium sp F28. In some embodiments the biocatalyst is Moraxella. In some embodiments the biocatalyst is Pantoea endophytica. In some embodiments the biocatalyst is Pantoea agglomerans. In some embodiments the biocatalyst is Pseudomonas chlororaphis. In some embodiments the biocatalyst is Pseudomonas putida. In some embodiments the biocatalyst is Rhizobium. In some embodiments the biocatalyst is Rhodopseudomonas palustris. In some embodiments the biocatalyst is Serratia liquefaciens, Serratia marcescens, Amycolatopsis, Arthrobacter, Brevibacterium sp CH1. In some embodiments the biocatalyst is Brevibacterium sp CH2. In some embodiments the biocatalyst is Brevibacterium sp R312. In some embodiments the biocatalyst is Brevibacterium imperia/e. In some embodiments the biocatalyst is Corynebacterium nitrilophilus. In some embodiments the biocatalyst is Corynebacterium pseudodiphteriticum. In some embodiments the biocatalyst is Corynebacterium glutamicum. In some embodiments the biocatalyst is Corynebacterium hoffmanii. In some embodiments the biocatalyst is Microbacterium imperia/e. In some embodiments the biocatalyst is Microbacterium smegmatis. In some embodiments the biocatalyst is Micrococcusluteus. In some embodiments the biocatalyst is Nocardia globerula. In some embodiments the biocatalyst is Nocardia rhodochrous. In some embodiments the biocatalyst is Pseudonocardia thermophila. In some embodiments the biocatalyst is Trichoderma. In some embodiments the biocatalyst is Myrothecium verrucaria. In some embodiments the biocatalyst is Aureobasidium pullulans. In some embodiments the biocatalyst is Candida famata. In some embodiments the biocatalyst is Candida guilliermondii. In some embodiments the biocatalyst is Candida tropicalis. In some embodiments the biocatalyst is Cryptococcus flavus. In some embodiments the biocatalyst is Cryptococcus sp UFMG-Y28. In some embodiments the biocatalyst is Debaryomyces hanseii. In some embodiments the biocatalyst is Geotrichum candidum. In some embodiments the biocatalyst is Geotrichum sp JR1. In some embodiments the biocatalyst is Hanseniaspora. In some embodiments the biocatalyst is Kluyveromyces thermotolerans. In some embodiments the biocatalyst is Pichia kluyveri. In some embodiments the biocatalyst is Rhodotorula glutinis. In some embodiments the biocatalyst is Escherichia coli. In some embodiments the biocatalyst is Geobacillus sp. In some embodiments the biocatalyst is RAPc8. In some embodiments the biocatalyst is Comomonas testosteroni. In some embodiments the biocatalyst is Pyrococcus abyssi. In some embodiments the biocatalyst is Pyrococcus furiosus. In some embodiments the biocatalyst is Pyrococcus horikoshii.

In a preferred embodiment of the present invention the biocatalyst is Rhodococcus rhodochrous. In some embodiments of the present invention the biocatalyst is of the strain Rhodococcus rhodochrous NCIMB 41164. In some embodiments of the present invention the biocatalyst is of the strain Rhodococcus rhodochrous J-1 (Accession number: FERM BP-1478). In some embodiments the biocatalyst is of the strain Rhodococcus rhodochrous M8 (Accession number: VKPMB-S 926). In some embodiments of the present invention the biocatalyst is of the strain. In some embodiments of the present invention the biocatalyst is of the strain Rhodococcus rhodochrous M33. In some embodiments of the present invention the biocatalyst is of the strain Rhodococcus pyridinovorans. In some embodiments of the present invention biocatalyst is of the strain Escherichia coli MT-10822 (Accession number: FERM BP-5785).

In another preferred embodiment of the present invention, the biocatalyst is Rhodococcus pyridinovorans.

According to the present invention, combinations of these microorganisms can be used as well. Further, the above microorganisms can be cultured by any method that is appropriate for a given microbial species. The microbial biocatalyst of the present invention that is prepared from microorganisms refers to a culture solution obtained by culturing microorganisms, cells obtained by a harvesting process or the like, cell disrupted by ultrasonication or the like, or those prepared after cell disruption including a crude enzyme, a partially-purified enzyme or a purified enzyme. A mode to use the microbial catalyst may be appropriately selected depending on enzyme stability, production scale and the like.

In some embodiments of the present invention, the biocatalyst used for converting acrylonitrile to acrylamide as described herein may be washed before the use in said reaction. In some embodiments, the biocatalyst may be washed once with water, a buffer or the like, and then washed with acrylic acid before the reaction. In some embodiments the biocatalyst used herein is washed with acrylic acid before the reaction as described in detail in EP1380652. In some embodiments the biocatalyst may be washed with acrylic acid immediately before the reaction. Further, any washing methods can be employed. Examples of such a method that can be applied according to the present invention include a method which involves repeated washing and centrifugation, and a washing method using a hollow fiber membrane. Further, immobilized biocatalysts can be washed by repeating agitation and precipitation of the immobilized catalysts in a wash and the removal of supernatant. Any washing method and any number of washing can be appropriately set in consideration of washing efficiency, enzyme stability and the like. The concentration of acrylic acid to be used for washing is preferably between 0.01% by mass and 10% by mass in an aqueous acrylic solution. More preferably, the concentration is between 0.05% by mass and 1% by mass, and most preferably is 0.1% by mass.

In other embodiments of the present invention, the biocatalyst used for converting acrylonitrile to acrylamide as described herein need not be washed before use in said reaction. In such embodiments the biocatalyst is not washed prior to use in the reaction. In this case, the other additives such as water and buffer solution would merely be added.

In context with the present invention, nitrile hydratase encoding microorganisms which are not naturally encoding nitrile hydratase may be genetically engineered microorganisms which naturally do not contain a gene encoding a nitrile hydratase but which have been manipulated such as to contain a polynucleotide encoding a nitrile hydratase (e.g., via transformation, transduction, transfection, conjugation, or other methods suitable to transfer or insert a polynucleotide into a cell as known in the art; cf. Sambrook and Russell 2001, Molecular Cloning: A Laboratory Manual, CSH Press, Cold Spring Harbor, N.Y., USA), thus enabling the microorganisms to produce and stably maintain the nitrile hydratase enzyme. For this purpose, it may further be required to insert additional polynucleotides which may be necessary to allow transcription and translation of the nitrile hydratase gene or mRNA, respectively. Such additional polynucleotides may comprise, inter alia, promoter sequences, polyT- or polyU-tails, or replication origins or other plasmid-control sequences. In this context, such genetically engineered microorganisms which naturally do not contain a gene encoding a nitrile hydratase but which have been manipulated such as to contain a polynucleotides encoding a nitrile hydratase may be prokaryotic or eukaryotic microorganisms.

Examples for such prokaryotic microorganisms include, e.g., representatives of the species Escherichia coli. Examples for such eukaryotic microorganisms include, e.g., yeast (e.g., Saccharomyces cerevisiae).

In context of the present invention, the term “nitrile hydratase” (also referred to herein as NHase) generally means an enzyme which is capable of catalyzing the conversion (i.e. hydration) of acrylonitrile to acrylamide. Such an enzyme may be, e.g., the enzyme registered under IUBMB nomenclature as of Apr. 1, 2014: EC 4.2.1.84; CAS-No. 2391-37-5. However, the term “nitrile hydratase” as used herein also encompasses modified or enhanced enzymes which are, e.g., capable of converting acrylonitrile to acrylamide more quickly, or which can be produced at a higher yield/time-ratio, or which are more stable, as long as they are capable to catalyze conversion (i.e. hydration) of acrylonitrile to acrylamide. Methods for determining the ability of a given biocatalyst (e.g., microorganism or enzyme) for catalyzing the conversion of acrylonitrile to acrylamide are known in the art. As an example, in context with the present invention, activity of a given biocatalyst to act as a nitrile hydratase in the sense of the present invention may be determined as follows: First reacting 100 μl of a cell suspension, cell lysate, dissolved enzyme powder or any other preparation containing the supposed nitrile hydratase with 875 μl of an 50 mM potassium phosphate buffer and 25 μl of acrylonitrile at 25° C. on an eppendorf tube shaker at 1,000 rpm for 10 minutes. After 10 minutes of reaction time, samples may be drawn and immediately quenched by adding the same volume of 1.4% hydrochloric acid. After mixing of the sample, cells may be removed by centrifugation for 1 minute at 10,000 rpm and the amount of acrylamide formed is determined by analyzing the clear supernatant by HPLC. For affirmation of an enzyme to be a nitrile hydratase in context with the present invention, the concentration of acrylamide shall be between 0.25 and 1.25 mmol/l—if necessary, the sample has to be diluted accordingly and the conversion has to be repeated. The enzyme activity may then be deduced from the concentration of acrylamide by dividing the acrylamide concentration derived from H PLC analysis by the reaction time, which has been 10 minutes and by multiplying this value with the dilution factor between HPLC sample and original sample. Activities >5 U/mg dry cell weight, preferably >25 U/mg dry cell weight, more preferably >50 U/mg dry cell weight, most preferably >100 U/mg dry cell weight indicate the presence of a functional biocatalyst and are considered as biocatalyst capable of converting acrylonitrile to acrylamide in context with the present invention.

In context with the present invention, the nitrile hydratase may be a polypeptide encoded by a polynucleotide which comprises or consists of a nucleotide sequence which is at least 70%, preferably at least 75%, more preferably at least 80%, more preferably at least 85%, more preferably at least 90%, more preferably at least 95%, more preferably at least 96%, more preferably at least 97%, more preferably at least 98%, more preferably at least 99%, more preferably at least 99.5%, and most preferably 100% identical to the nucleotide sequence of SEQ ID NO: 1 (alpha-subunit of nitrile hydratase of Rhodococcus rhodochrous: GTGAGCGAGCACGTCAATAAGTACACGGAGTACGAGGCACGTACCAAGGCGATCGAAACC TTGCTGTACGAGCGAGGGCTCATCACGCCCGCCGCGGTCGACCGAGTCGTTTCGTACTAC GAGAACGAGATCGGCCCGATGGGCGGTGCCAAGGTCGTGGCCAAGTCCTGGGTGGACCC TGAGTACCGCAAGTGGCTCGAAGAGGACGCGACGGCCGCGATGGCGTCATTGGGCTATG CCGGTGAGCAGGCACACCAAATTTCGGCGGTCTTCAACGACTCCCAAACGCATCACGTGG TGGTGTGCACTCTGTGTTCGTGCTATCCGTGGCCGGTGCTTGGTCTCCCGCCCGCCTGGT ACAAGAGCATGGAGTACCGGTCCCGAGTGGTAGCGGACCCTCGTGGAGTGCTCAAGCGC GATTTCGGTTTCGACATCCCCGATGAGGTGGAGGTCAGGGTTTGGGACAGCAGCTCCGAA ATCCGCTACATCGTCATCCCGGAACGGCCGGCCGGCACCGACGGTTGGTCCGAGGAGGA GCTGACGAAGCTGGTGAGCCGGGACTCGATGATCGGTGTCAGTAATGCGCTCACACCGCA GGAAGTGATCGTATGA) and/or to the nucleotide sequence of SEQ ID NO: 3 (beta-subunit of nitrile hydratase of Rhodococcus rhodochrous: ATGGATGGTATCCACGACACAGGCGGCATGACCGGATACGGACCGGTCCCCTATCAGAAG GACGAGCCCTTCTTCCACTACGAGTGGGAGGGTCGGACCCTGTCAATTCTGACTTGGATG CATCTCAAGGGCATATCGTGGTGGGACAAGTCGCGGTTCTTCCGGGAGTCGATGGGGAAC GAAAACTACGTCAACGAGATTCGCAACTCGTACTACACCCACTGGCTGAGTGCGGCAGAA CGTATCCTCGTCGCCGACAAGATCATCACCGAAGAAGAGCGAAAGCACCGTGTGCAAGAG ATCCTTGAGGGTCGGTACACGGACAGGAAGCCGTCGCGGAAGTTCGATCCGGCCCAGAT CGAGAAGGCGATCGAACGGCTTCACGAGCCCCACTCCCTAGCGCTTCCAGGAGCGGAGC CGAGTTTCTCTCTCGGTGACAAGATCAAAGTGAAGAGTATGAACCCGCTGGGACACACAC GGTGCCCGAAATATGTGCGGAACAAGATCGGGGAAATCGTCGCCTACCACGGCTGCCAGA TCTATCCCGAGAGCAGCTCCGCCGGCCTCGGCGACGATCCTCGCCCGCTCTACACGGTC GCGTTTTCCGCCCAGGAACTGTGGGGCGACGACGGAAACGGGAAAGACGTAGTGTGCGT CGATCTCTGGGAACCGTACCTGATCTCTGCGTGA), provided that the polypeptide encoded by said polynucleotide is capable of catalyzing hydration of acrylonitrile to acrylamide (i.e. has nitrile hydratase activity) as described and exemplified herein. Also in the context with the present invention, the nitrile hydratase may be a polypeptide which comprises or consists of an amino acid sequence which is at least 70%, preferably at least 75%, more preferably at least 80%, more preferably at least 85%, more preferably at least 90%, more preferably at least 95%, more preferably at least 96%, more preferably at least 97%, more preferably at least 98%, more preferably at least 99%, more preferably at least 99.5%, and most preferably 100% identical to the amino acid sequence of SEQ ID NO: 2 (alpha-subunit of nitrile hydratase of Rhodococcus rhodochrous: VSEHVNKYTE YEARTKAIET LLYERGLITP AAVDRVVSYY ENEIGPMGGA KVVAKSWVDP EYRKWLEEDA TAAMASLGYA GEQAHQISAV FNDSQTHHVV VCTLCSCYPW PVLGLPPAWY KSMEYRSRVV ADPRGVLKRD FGFDIPDEVE VRVWDSSSEI RYIVIPERPA GTDGWSEEEL TKLVSRDSMI GVSNALTPQE VIV) and/or to the amino acid sequence of SEQ ID NO: 4 (beta-subunit of nitrile hydratase of R. rhodochrous: MDGIHDTGGM TGYGPVPYQK DEPFFHYEWE GRTLSILTWM HLKGISWWDK SRFFRESMGN ENYVNEIRNSY YTHWLSAAE RILVADKIIT EEERKHRVQE ILEGRYTDRK PSRKFDPAQI EKAIERLHEP HSLALPGAEP SFSLGDKIKV KSMNPLGHTR CPKYVRNKIG EIVAYHGCQI YPESSSAGLG DDPRPLYTVA FSAQELWGDD GNGKDVVCVD LWEPYLISA), provided that said polypeptide is capable of catalyzing hydration of acrylonitrile to acrylamide as described and exemplified herein.

The level of identity between two or more sequences (e.g., nucleic acid sequences or amino acid sequences) can be easily determined by methods known in the art, e.g., by BLAST analysis. Generally, in context with the present invention, if two sequences (e.g., polynucleotide sequences or amino acid sequences) to be compared by, e.g., sequence comparisons differ in identity, then the term “identity” may refer to the shorter sequence and that part of the longer sequence that matches said shorter sequence. Therefore, when the sequences which are compared do not have the same length, the degree of identity may preferably either refer to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that matches the shorter sequence. Furthermore, as used herein, identity levels of nucleic acid sequences or amino acid sequences may refer to the entire length of the respective sequence and is preferably assessed pair-wise, wherein each gap is to be counted as one mismatch. These definitions for sequence comparisons (e.g., establishment of “identity” values) are to be applied for all sequences described and disclosed herein.

Moreover, the term “identity” as used herein means that there is a functional and/or structural equivalence between the corresponding sequences. Nucleic acid/amino acid sequences having the given identity levels to the herein-described particular nucleic acid/amino acid sequences may represent derivatives/variants of these sequences which, preferably, have the same biological function. They may be either naturally occurring variations, for instance sequences from other varieties, species, etc., or mutations, and said mutations may have formed naturally or may have been produced by deliberate mutagenesis. Furthermore, the variations may be synthetically produced sequences. The variants may be naturally occurring variants or synthetically produced variants or variants produced by recombinant DNA techniques. Deviations from the above-described nucleic acid sequences may have been produced, e.g., by deletion, substitution, addition, insertion and/or recombination. The term “addition” refers to adding at least one nucleic acid residue/amino acid to the end of the given sequence, whereas “insertion” refers to inserting at least one nucleic acid residue/amino acid within a given sequence. The term “deletion” refers to deleting or removal of at least one nucleic acid residue or amino acid residue in a given sequence. The term “substitution” refers to the replacement of at least one nucleic acid residue/amino acid residue in a given sequence. Again, these definitions as used here apply, mutatis mutandis, for all sequences provided and described herein.

Generally, as used herein, the terms “polynucleotide” and “nucleic acid” or “nucleic acid molecule” are to be construed synonymously. Generally, nucleic acid molecules may comprise inter alia DNA molecules, RNA molecules, oligonucleotide thiophosphates, substituted ribo-oligonucleotides or PNA molecules. Furthermore, the term “nucleic acid molecule” may refer to DNA or RNA or hybrids thereof or any modification thereof that is known in the art (see, e.g., U.S. Pat. Nos. 5,525,711, 4,711,955, 5,792,608 or EP 302175 for examples of modifications). The polynucleotide sequence may be single- or double-stranded, linear or circular, natural or synthetic, and without any size limitation. For instance, the polynucleotide sequence may be genomic DNA, cDNA, mitochondrial DNA, mRNA, antisense RNA, ribozymal RNA or a DNA encoding such RNAs or chimeroplasts (Gamper, Nucleic Acids Research, 2000, 28, 4332-4339). Said polynucleotide sequence may be in the form of a vector, plasmid or of viral DNA or RNA. Also described herein are nucleic acid molecules which are complementary to the nucleic acid molecules described above and nucleic acid molecules which are able to hybridize to nucleic acid molecules described herein. A nucleic acid molecule described herein may also be a fragment of the nucleic acid molecules in context of the present invention. Particularly, such a fragment is a functional fragment. Examples for such functional fragments are nucleic acid molecules which can serve as primers.

When adding the biocatalyst to the reactor in any one of the methods of the present invention, the biocatalyst may be taken directly from the fermentation broth. Alternatively, the biocatalyst may be dried before. With this respect, the biocatalyst may be dried before being added to the reactor according to any one of the methods of the present invention. In this context the term “before” does not necessarily mean that the biocatalyst has been dried and is then immediately added to the reactor. It is rather sufficient that the biocatalyst has undergone a drying step at any time before it is added to the reactor, independently of whether further steps between the drying and the addition are performed or not. As non-limiting examples, such further steps between the drying step and the addition to the reactor may be storage or reconstitution. However, it is also possible to add the biocatalyst to the reactor directly after drying. The inventors have found that by using a biocatalyst, which has undergone a drying step, the concentration of acrylic acid in an aqueous acrylamide solution obtained by any one of the methods described herein is further reduced in comparison to the case that a biocatalyst is used which has not undergone drying before being employed in the bioconversion.

The present invention also includes a biocatalyst composition obtainable by the inventive method of storing the biocatalyst, including any of the preferred embodiments recited herein.

The present invention also incorporates the use of the aforesaid biocatalyst composition, and specifically the biocatalyst composition resulting from the inventive method of storing a biocatalyst composition, for preparing an aqueous solution of (meth-) acrylamide in a process of converting (meth-) acrylonitrile to (meth-) acrylamide.

The biocatalyst composition produced in accordance with the aforementioned biocatalyst storage method may easily be restored as a ready to use biocatalyst composition. This may be achieved by first increasing the temperature and reducing the concentration of the of the stored biocatalyst composition containing the biocatalyst.

In one desirable embodiment of the present invention we provide a method in which the inventive method of storing a biocatalyst is extended to restoring the biocatalyst following a period of storage and then employing the restored biocatalyst in a process of producing an aqueous acrylamide solution.

Thus, in a further aspect of the present invention we provide a method of producing an aqueous (meth-) acrylamide solution from (meth-) acrylonitrile comprising the steps of:

(a) providing an aqueous suspension comprising the biocatalyst, which is capable of converting acrylonitrile to acrylamide, which aqueous suspension is an aqueous fermentation broth;

(b) sequentially in either order or simultaneously
(b1) concentrating the aqueous suspension comprising the biocatalyst to a concentration of at least 3% (w/w); and
(b2) reducing the temperature of the aqueous suspension comprising the biocatalyst to a temperature of below 8° C.,

thereby forming a concentrated aqueous suspension;

(c) maintaining the concentrated aqueous suspension of step (b) at a temperature of below 8° C.;

(d) providing a ready to use aqueous biocatalyst composition from the aqueous suspension of step (c) by
(i) increasing the temperature of the concentrated aqueous biocatalyst composition to a temperature of at least 8° C., preferably at least 15° C.; and
(ii) diluting the concentration of the aqueous biocatalyst composition to a concentration of below 3% (w/w);

in which (i) and (ii) are conducted simultaneously or sequentially in either order;

(e) contacting (meth-) acrylonitrile in an aqueous medium with the ready to use aqueous biocatalyst composition; and

(f) conducting the conversion reaction of (meth-) acrylonitrile to produce the aqueous (meth-) acrylamide solution.

In addition, this method desirably includes any of the aforementioned specific embodiments described herein.

In step (d) the ready to use aqueous biocatalyst composition containing the biocatalyst is provided. Parts (i) and (ii) of step (e) may be carried out simultaneously or sequentially in either order.

This may be achieved by first increasing the temperature of the of the concentrated biocatalyst composition in accordance with part (i). Desirably, the concentrated biocatalyst composition may be increased to a more ambient temperature by subjecting it to an environment at a temperature of from 15° C. to 30° C., suitably from 20° C. to 25° C. The desired target temperature for the biocatalyst may often depend on the individual microorganism, for instance the optimum temperature for the microorganism containing the enzyme capable of converting (meth-) acrylonitrile to (meth-) acrylamide. Typically, the target temperature for the restored biocatalyst composition may be from 15° C. to 25° C., such as from 17° C. to 23° C., particularly about 20° C.

Part (ii) of step (d) may desirably be achieved by adding water or aqueous liquid to the concentrated biocatalyst composition once the temperature has been raised to the desired temperature in accordance with Part (i). It may also be desirable to adjust the pH of the biocatalyst composition. This could be done simultaneously with or as part of the dilution of the concentrated biocatalyst composition.

The adjustment of the pH of the biocatalyst composition may be carried out before, during or after carrying out step (e). The specific adjustment of the pH may often depend upon the pH that would provide the optimum activity for a given biocatalyst composition, for instance as the optimum conditions for the microorganism containing the biocatalyst. Typically, the desired pH may be optimally about neutral, for instance a pH discussed previously, such as a pH from 5.5 to 8.5, desirably a pH from 6.5 to 7.5 and desirably around pH 7. The pH may be maintained by use of a suitable pH buffer. For instance, where the pH is to be maintained at pH 7.0 a potassium phosphate buffer may be used. Other suitable pH buffers are known to those skilled in the art, especially from the patents and literature. As also described previously the biocatalyst composition may be stored at a more acidic pH, for instance from pH 4.5 to below 7.0, or for instance from pH 5 to 6.5. This may be achieved by adding a particular buffer solution to the biocatalyst composition, said buffer solution capable of buffering the biocatalyst composition to the desired pH.

Dilution of the concentrated biocatalyst composition may also be achieved at least in part by combining the concentrated biocatalyst composition from part (i) with water and (meth-) acrylonitrile either separately or as a mixture, for instance in a reaction vessel for conducting the conversion of the (meth-) acrylonitrile to (meth-) acrylamide. Thus, in this form step (d) part (ii) may be done simultaneously with contacting the (meth-) acrylonitrile with the ready to use aqueous biocatalyst composition according to step (e).

Carrying out the reaction of converting the (meth-) acrylonitrile to (meth-) acrylamide may be carried out in accordance with known process parameters described in the patents and literature. This may for instance be in accordance with any of EP 1385972, EP 2267143, EP 2518154, EP 2336346, JP 2015057968, JP 2014176344, WO 2016/006556, or WO 2017167803.

Any one of the methods described herein may be carried out using a continuous process. In particular, the term “continuous process” as used herein refers to a method, wherein an aqueous acrylamide solution is produced in a continuous manner without collecting the entire reaction mixture in the reactor. This means that the raw materials for the reaction, which may comprise the biocatalyst, water and acrylonitrile, are fed to the reactor continuously or intermittently and that the obtained product is recovered from the reactor continuously or intermittently.

Alternatively, any one of the methods of the present invention may be carried out using a semi-batch process. In particular, the term “semi-batch process” as used herein may comprise that an aqueous acrylamide solution is produced in a discontinuous manner. According to a non-limiting example for carrying out such a semi-batch process water, a certain amount of acrylonitrile and the biocatalyst are placed in a reactor. Further acrylonitrile is then added during the bioconversion until a desired content of acrylamide of the composition is reached. After such desired content of acrylamide is reached, the obtained composition is entirely recovered from the reactor, before new reactants are placed therein.

Typically, the (meth-) acrylonitrile, water and the biocatalyst composition provided from step (d) may be combined or transferred to a reaction vessel. Desirably, the reaction mixture may be mixed or stirred in the reaction vessel by means of a suitable agitating or mixing means. The reaction may be monitored using suitable instrumentation, for instance an FTIR instrument used to detect (meth-) acrylonitrile and (meth-) acrylamide concentrations over the course of the reaction. It may be desirable to maintain a constant concentration of the (meth-) acrylonitrile during the reaction.

In general, in any one of the methods of the present invention the acrylonitrile content and/or the acrylamide content during step (f) may be measured using Fourier Transform Infrared Spectroscopy (FTIR). In particular, the acrylonitrile content and/or the acrylamide content may be measured online using FTIR.

Regarding the feeding of acrylonitrile during the bioconversion step (f), according to a non-limiting embodiment of any one of the methods of the present invention the acrylonitrile may be fed such that the content of acrylonitrile during step (f) is maintained within a range of ±10 w/w %, preferably of ±5 w/w %, more preferably of ±2 w/w %, most preferably of ±1 w/w % of a predetermined value of the acrylonitrile content, wherein the indications of w/w % are each referred to the total weight of acrylonitrile in the reactor. In particular, in any one of the methods of the present invention the acrylonitrile may be fed such that the content of acrylonitrile during step (f) is maintained substantially constant at a predetermined value.

In one embodiment of the present invention the acrylonitrile concentration may suitably be a concentration of from 0.2 to 4% (w/v), for instance from 0.5 to 3.5% (w/w), for instance from 1 to 3% (w/w) at least during part of the reaction. Desirably, it may be desirable to vary the concentration during the conversion of (meth-) acrylonitrile to (meth-) acrylamide. For instance, the concentration of (meth-) acrylonitrile earlier in the reaction may be higher than the (meth-) acrylonitrile concentration as the reaction proceeds towards completion. In one preferred form, the (meth-) acrylonitrile concentration for the first 20 minutes to 2 hours of the reaction may be conducted at a (meth-) acrylonitrile concentration of from 0.75 to 4% (w/w), suitably from 1.0 to 3.5% (w/w), desirably from 1.5 to 3.0% (w/w) and then the remainder of the reaction conducted at a (meth-) acrylonitrile concentration of from 0.2 to 2.0% (w/w), suitably from 0.5 to 1.5% (w/w), desirably from 0.5 to 1.2% (w/w).

Typically, the reaction may desirably be maintained at a relatively constant temperature during its course. This may be achieved by customary methods of maintaining an isothermal condition described in the literature and patents. In one suitable method, the reaction may be maintained under isothermal conditions by pumping the reaction mixture through an external cooling loop.

Suitably, such an external cooling loop should be connected to the reaction vessel in which the reaction takes place. Typical isothermal conditions may be an ambient temperature which, for instance, lies between 15 and 30° C., desirably between 20 and 28° C. Preferably, the reaction temperature should not vary by more than ±5° C., ideally by not more than ±2° C. The reaction may be continued until the desired concentration of (meth-) acrylamide is achieved. Suitably, the reaction may be continued until the concentration of (meth-) acrylamide has reached at least 45% (w/w), preferably at least 48% (w/w), more preferably at least 50% (w/w), more preferably still above 51% (w/w). At the point where the (meth-) acrylamide concentration has reached the desired level the (meth-) acrylonitrile feed to the reaction vessel would typically be stopped.

The present invention also includes an aqueous (meth-) acrylamide solution which is obtainable by this inventive method.

The aqueous (meth-) acrylamide solution obtained in this inventive method may be used in a subsequent polymerisation reaction, either alone in order to manufacture the homopolymer of (meth-) acrylamide or together with one or more other ethylenically unsaturated monomers to produce copolymers of (meth-) acrylamide, which are generally described as polyacrylamides.

In one desirable embodiment of the present invention we provide a method in which the inventive method of storing a biocatalyst is extended to restoring the biocatalyst following a period of storage and then employing the restored biocatalyst in a process of producing an aqueous acrylamide solution and then polymerising the aqueous acrylamide solution alone or in conjunction with one or more ethylenically unsaturated monomers to produce a polyacrylamide.

Thus, in a further aspect of the present invention we provide a method of producing a polyacrylamide comprising the steps of:

(a) providing an aqueous suspension comprising the biocatalyst, which is capable of converting acrylonitrile to acrylamide, which aqueous suspension is an aqueous fermentation broth;

(b) sequentially in either order or simultaneously
(b1) concentrating the aqueous suspension comprising the biocatalyst to a concentration of at least 3% (w/w); and
(b2) reducing the temperature of the aqueous suspension comprising the biocatalyst to a temperature of below 8° C.,

thereby forming a concentrated aqueous suspension;

(c) maintaining the concentrated aqueous suspension of step (b) at a temperature of below 8° C.;

(d) providing a ready to use aqueous biocatalyst composition from the concentrated aqueous suspension of step (c) by
(i) increasing the temperature of the concentrated aqueous biocatalyst composition to a temperature of at least 8° C., preferably at least 15° C.; and
(ii) diluting the concentration of the aqueous biocatalyst composition to a concentration of below 3% (w/w);

in which (i) and (ii) are conducted simultaneously or sequentially in either order;

(e) contacting (meth) acrylonitrile in an aqueous medium with the ready to use aqueous biocatalyst composition;

(f) conducting the conversion reaction of (meth-) acrylonitrile to produce the aqueous (meth-) acrylamide solution; and

(g) polymerising the aqueous (meth-) acrylamide solution obtained in step (f) to polyacrylamide.

Such polymerisation of the aqueous (meth-) acrylamide may be carried out according to any of the methods of polymerisation known to those skilled in the art and well-documented in the patents and literature. Typically, the aqueous solution of (meth-) acrylamide may be subjected to polymerisation conditions, for instance by the introduction of initiators and optionally chain transfer agents. Suitable initiators include, for instance, redox initiators and/or thermal initiators. Examples of suitable redox initiators include redox couples comprising oxidising agents, such as peroxides (e.g. tertiary butyl hydroperoxide, persulphates and other peroxy compounds) in combination with reducing agents, such as sulphites or ferrous compounds (e.g. ferrous ammonium sulphate). Examples of thermal initiators may include azo compounds such as 2,2′-azobis(isobutyronitrile), 2,2′-azobis(4-methoxy-2,4-dimethylvaleronitrile), 4,4′-azobis(4-cyanovaleric acid), etc. Examples of suitable chain transfer agents include mercapto compounds, such as 2-mercapto ethanol; and hypophosphite compounds such as sodium hypophosphite.

The biocatalyst may optionally be removed from the aqueous (meth-) acrylamide solution prior to carrying out the polymerisation.

Nevertheless, as disclosed in WO 2005/054488, it may be desired that the biocatalyst is not substantially removed from the aqueous acrylamide solution of the completion of the conversion of (meth-) acrylonitrile to (meth-) acrylamide. This document rather describes a process for preparing a polymer solution, wherein the monomer contains cellular material and/or components of the fermentation broth. Such polymers seem to have even specifically designed features and properties, without the need for removing either the biocatalyst or the fermentation broth. Further, the presence of biocatalyst in the acrylamide solution may also be seen as desirable, as reported by EP 2264003 and US 2011006258.

However, there are certain applications of polyacrylamides where it may be necessary to remove the biocatalyst. For instance, it may be required that polyacrylamides for use in tertiary oil recovery do not contain biological material. In the case of tertiary oil recovery rock layer injectability of the aqueous polyacrylamide solution is of particular relevance when introducing the polymer solution into the ground. More precisely, when using the biological synthesis method for acrylamide preparation, the presence of cells of the biocatalyst having a size of similar magnitude to the rock pores should be avoided so as not to block the rock pores which could affect the performance of the polymer solution as a tertiary oil recovery aid. Further, there may be other applications where the presence of biocatalyst cells would be undesirable in the polyacrylamide product. It would not be practical or economically viable to separate the biocatalyst from the polyacrylamide after polymerisation.

Suitable methods for separation of the biocatalyst are known in the art and include, for example, centrifugation, sedimentation (e.g., with flocculation), membrane separation and filtration.

WO 02/088372 describes, for instance, a method and device for separating biocatalyst from the acrylamide solution using a tube centrifuge or an annular gap centrifuge, optionally in combination with flocculation. The biocatalyst is washed with water to remove residual monomer and then used in the next bioconversion reaction.

Removal of the biocatalyst after completion of the conversion of acrylonitrile to acrylamide can be carried out by diverse filtration techniques, as described in for instance EP 2019146 and CN 203319905, each with the target to remove impurities from the acrylamide reaction solution.

Separation of the biocatalyst from an aqueous acrylamide solution prior to polymerisation is described in WO 2017/167803 with the objective of producing a polyacrylamide solution having increased viscosity when compared to a reference solution. In a preferred embodiment employs disk stack separation performed with a specific settling area of 19.67 m²h/l or more.

Generally, the present invention relates to all embodiments described herein as well as all permutations and combinations thereof. Any particular aspects or embodiments described herein must not be construed as limiting the scope of the present invention on such aspects or embodiments.

The following examples further describe and exemplify the invention provided herein without limiting the invention to any specifications or embodiments described therein.

### EXAMPLES

### Example 1

Forming Stored Biocatalyst The Rhodococcus rhodocrous strain NCIMB 41164 was cultivated for expression of nitrile hydratase that catalyzes the bioconversion of acrylonitrile (ACN) to acrylamide (ACM). After the fermentation, the fermentation broth was concentrated by a disk stack plate separator to a total solids content of approximately 16% (w/w) and the pH was adjusted to pH ˜7.2 using aqueous solutions of ammonia or phosphoric acid, respectively, depending to the starting pH. The specific settling area was 40 m²h/l. A portion of this suspension was subjected to centrifugation followed by resuspension in a fraction of the supernatant giving a suspension with a total solids content of approximately 23% (w/w). These suspensions were stored in aliquots of approximately 15 g (in 50 mL polypropylene tubes) at −20° C. or 7-8° C., respectively.

The total dry mass (total solids content) was determined as follows.

The determination of the total dry mass was performed in the BASF laboratories in Ludwigshafen, Germany using a Mettler Toledo HR73 Halogen Moisture Analyser using the following procedure:

| Step | Procedure |
|---|---|
| 1 | Open HR 73 tray holder and insert an empty tray and |
| | fibreglass pad. |
| 2 | Close the tray holder and push “Tara”-Button. |
| 3 | Open the tray holder again and add 1.2 to 1.4 g of |
| | analyte on the pad. Spread the sample on the complete |
| | pad. Close the tray holder. |
| 4 | Choose “Prg. 3” if it is not selected by default. |
| | Program descriptor: constant temperature of 130° C. until |
| | constant weight for 30 seconds. |
| 5 | Push “Start”-Button. |
| 6 | Wait until the measurement is complete. Then open tray |
| | holder and dispose of the pad with the dried sample. |

Restoring Biocatalyst and Converting Acrylonitrile to Acrylamide

On the day of the experiment, samples were thawed at 25° C. in a rotary shaker. 4.5 g (for samples with 23% (w/w) total solids in concentrate) or 6.6 g (for samples with 16% (w/w) total solids in concentrate) of biocatalyst concentrate were filled up to 30 mL by adding a 100 mM potassium phosphate buffer (pH 7.0) and the resulting suspension was mixed by inverting the tube several times. The resulting biocatalyst slurry was transferred to the reaction vessel that contained 2397 g water and 55 g ACN pre-warmed to 26° C. to start the bioconversion of ACN to ACM. The reaction vessel was stirred with an overhead-stirrer at 250 rpm and ACN was continuously fed to the reaction mixture via a peristaltic pump. The entire reaction mixture was pumped through an external cooling loop connected to the reaction vessel to remove the heat of reaction and to maintain the temperature in the reaction vessel at 26° C. A FTIR instrument was used to detect ACN and ACM concentrations over the time course of the reaction. The FTIR signal was used to control the peristaltic pump that maintains the ACN concentration in the reaction mixture at 2% (w/w) during the first hour of reaction and at 0.8% (w/v) for the remainder of the reaction. The ACN feed was stopped after the ACM concentration reached the desired level of >51% (w/w). The results were interpreted by calculation of the initial ACN conversion rate normalized to the amount of catalyst (total solids content) added and the dosing time required to reach a threshold ACM concentration of >51% (w/w).

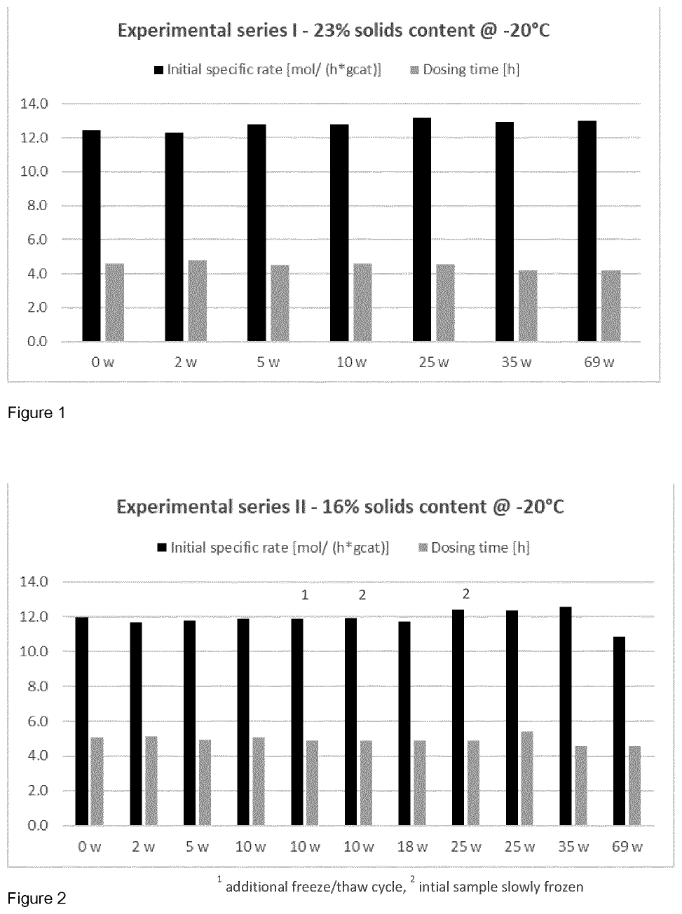

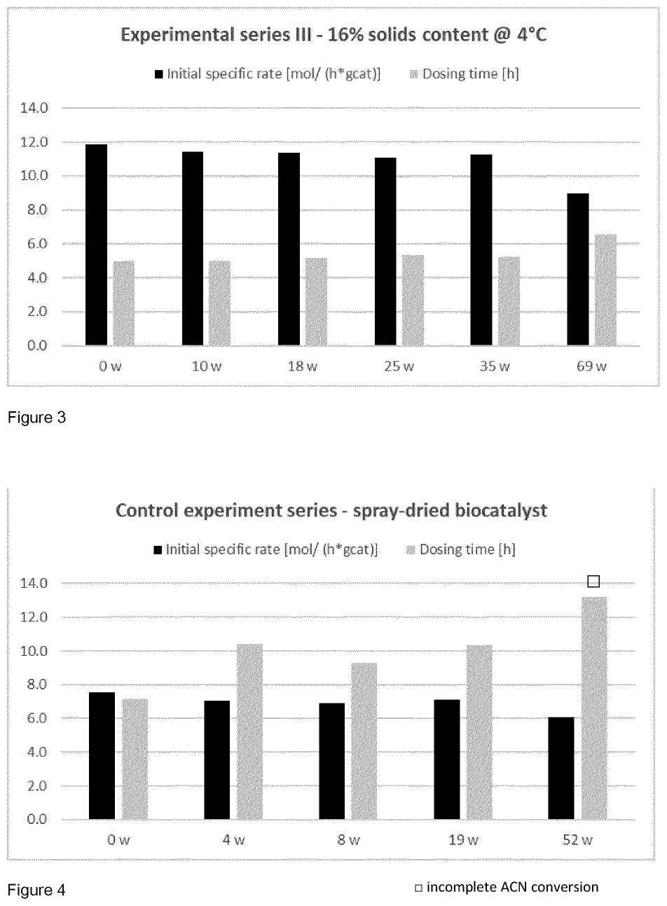

The results of storage capabilities of the respective biocatalyst compositions are illustrated in FIGS. 1-4. FIG. 1 illustrates Experiment Series I in which the stored biocatalyst has a solids of 23% (w/w) and a storage temperature of −20° C. The results showed that there is no change in initial rate and dosing time up to 69 weeks. FIG. 2 shows Experiment Series II in which the stored biocatalyst has a solids of 16% (w/w) and a storage temperature of −20° C. The results showed that there is no change in initial rate and dosing time up to 35 weeks. There is a slight reduction of initial rate after 69 weeks. FIG. 3 concerns Experiment Series III in which the stored biocatalyst has a solids of 16% (w/w) and a storage temperature of 4° C. The results indicate that there is no change in initial rate and dosing time up to 35 weeks. There is a slight reduction of initial rate an increased dosing time after 69 weeks. FIG. 4 relates to the Control Experiment Series employing spray dried biocatalyst. The results show a reduction in initial rate and a significant increase in dosing time. Significantly, the results show an incomplete acrylonitrile conversion after 52 weeks.

### Example 2

Experiments 1-3

50 g biocatalyst were transferred to a stirred reactor containing 561.7 g deionized water at room temperature. The temperature was adjusted to 20° C., and this temperature was controlled throughout the complete bioconversion reaction. 388.4 g acrylonitrile was added to the reactor at a constant rate of 194.2 g/h, so that the complete acrylonitrile amount was added within 2 hours. 22 hours after the feed had stopped (i.e. after 24 hours total reaction time), a sample was taken from the reaction solution and the acrylamide concentration was analyzed by HPLC. The results are shown in Table 1.

Experiments 4-6

6.3 g biocatalyst was suspended in water (total volume 50 mL) and gently mixed at room temperature for 10-20 minutes. The biocatalyst suspension was then transferred to a stirred reactor containing 561.7 g deionized water at room temperature. The temperature was adjusted to 20° C., and this temperature was controlled throughout the complete bioconversion reaction. 388.4 g acrylonitrile was added to the reactor at a constant rate of 194.2 g/h, so that the complete acrylonitrile amount was added within 2 hours. 22 hours after the feed had stopped (i.e. after 24 hours total reaction time), a sample was taken from the reaction solution and the acrylamide concentration was analyzed by HPLC. The results are shown in Table 1.

TABLE 1

| Exp. | | 1 | 2 | 1 | 3 |
|---|---|---|---|---|---|
| # | Resuspension medium | Day | weeks | month | months |
| 1 | 20° C., fermentation | 52% | 27% | 20% | 23% |
| | broth* | | | | |
| 2 | 4° C., fermentation | 52% | 52% | 52% | 27% |
| | broth* | | | | |
| 3 | −20° C., fermentation | 52% | 39% | 25% | 23% |
| | broth* | | | | |
| 4 | 20° C., fermentation | 52% | 52% | 46% | 39% |
| | concentrate** | | | | |
| 5 | 4° C., fermentation | 52% | 52% | 52% | 40% |
| | concentrate** | | | | |
| 6 | −20° C., fermentation | 52% | 52% | 52% | 51% |
| | concentrate** | | | | |

*Fermentation broth contains 3.8 g/L biocatalyst.
**Fermentation concentrate was prepared by concentrating the fermentation broth to a final concentration of 30 g/L biomass.

As shown in Table 1, conversion of acrylonitrile to acrylamide was significantly improved when the fermentation broth was concentrated to a final concentration of at least 3% solids (Exp. 4-6). In all the cases, the concentrated biocatalyst suspension has led to higher acrylamide concentration compared to the fermentation broth (Exp. 1-6).

The storage temperature should also be lower than 5° C. (maybe 10° C.) to improve the biocatalyst stability (Exp. 4-6).

1.-20. (canceled)

21. A method of storing a biocatalyst, which biocatalyst is capable of converting acrylonitrile to acrylamide, comprising the steps:
(a) providing an aqueous suspension comprising the biocatalyst, which is capable of converting acrylonitrile to acrylamide, which aqueous suspension is an aqueous fermentation broth;
(b) sequentially in either order or simultaneously
(b1) concentrating the aqueous suspension comprising the biocatalyst to a concentration of at least 3% (w/w); and
(b2) reducing the temperature of the aqueous suspension comprising the biocatalyst to a temperature of below 8° C.,
thereby forming a concentrated aqueous suspension; and
(c) maintaining the concentrated aqueous suspension of step (b) at a temperature of below 8° C.

22. A method according to claim 21 in which in step (b) the aqueous suspension is concentrated to a concentration of at least 10% (w/w), preferably at least 15% (w/w), more preferably at least 20% (w/w).

23. A method according to claim 21 in which in step (b) the aqueous suspension is concentrated to a concentration of from 3 to 60% (w/w), preferably from 5 to 50% (w/w), more preferably from 8 to 40% (w/w), still more preferably from 10 to 30% (w/w).

24. A method according to claim 21 in which the concentration step (b) comprises separating the biocatalyst as biocatalyst solids from the aqueous medium of the aqueous suspension followed by resuspension in an aqueous medium at a concentration of at least 3% (w/w).

25. A method according to claim 21 in which the concentrating of the aqueous suspension in step (b) employs one or more of centrifugation or filtration.

26. A method according to claim 25 in which the centrifugation is performed by disk stack separation.

27. A method according to claim 21 in which the concentration step (b) is achieved by disk stack separation performed with a specific settling area of 118.0 m²h/l or less, preferably 60 m²h/l or less, more preferably 40 m²h/l or less

28. A method according to claim 21 in which the temperature in steps (b) and (c) is up to 5° C., suitably from −25° C. to 5° C.

29. A method according to claim 21 in which the biocatalyst is a biocatalyst having nitrile hydratase activity.

30. A method according to claim 29 in which the biocatalyst nitrile hydratase nitrile hydratase activity and is selected from the group consisting of microorganisms belonging to Rhodococcus, Aspergillus, Acidovorax, Agrobacterium, Bacillus, Bradyrhizobium, Burkholderia, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Escherichia, Geobacillus, Comomonas, and Pyrococcus, and transformed microbial cells in which a nitrile hydratase gene is introduced.

31. A method according to claim 30, wherein the biocatalyst is selected from the group consisting of Rhodococcus, Pseudomonas, Escherichia and Geobacillus.

32. A method according to claim 29, wherein the microorganism is one selected from the group consisting of the species Rhodococcus rhodochrous, Rhodococcus erythropolis, Rhodococcus equi, Rhodococcus ruber, Rhodococcus opacus, Rhodococcus pyridinovorans, Aspergillus niger, Acidovorax avenae, Acidovorax facilis, Agrobacterium tumefaciens, Agrobacterium radiobacter, Bacillus subtilis, Bacillus pallidus, Bacillus smithii, Bacillus sp BR449, Bradyrhizobium oligotrophicum, Bradyrhizobium diazoefficiens, Bradyrhizobium japonicum, Burkholderia cenocepacia, Burkholderia gladioli, Klebsiella oxytoca, Klebsiella pneumonia, Klebsiella variicola, Mesorhizobium ciceri, Mesorhizobium opportunistum, Mesorhizobium sp F28, Moraxella, Pantoea endophytica, Pantoea agglomerans, Pseudomonas chlororaphis, Pseudomonas putida, Rhizobium, Rhodopseudomonas palustris, Serratia liquefaciens, Serratia marcescens, Amycolatopsis, Arthrobacter, Brevibacterium sp CH1, Brevibacterium sp CH2, Brevibacterium sp R312, Brevibacterium imperiale, Corynebacterium nitrilophilus, Corynebacterium pseudodiphteriticum, Corynebacterium glutamicum, Corynebacterium hoffmanii, Microbacterium imperiale, Microbacterium smegmatis, Micrococcus luteus, Nocardia globerula, Nocardia rhodochrous, Pseudonocardia thermophila, Trichoderma, Myrothecium verrucaria, Aureobasidium pullulans, Candida famata, Candida guilliermondii, Candida tropicalis, Cryptococcus flavus, Cryptococcus sp UFMG-Y28, Debaryomyces hanseii, Geotrichum candidum, Geotrichum sp JR1, Hanseniaspora, Kluyveromyces thermotolerans, Pichia kluyveri, Rhodotorula glutinis, Escherichia co/i, Geobacillus sp. RAPc8, Comomonas testosteroni, Pyrococcus abyssi, Pyrococcus furiosus, and Pyrococcus horikoshii.

33. A method according to claim 32, wherein the Rhodococcus rhodochrous biocatalyst is selected from Rhodococcus rhodochrous NCINMB 41164, Rhodococcus rhodochrous J-1 (Accession number: FERM BP-1478), Rhodococcus rhodochrous M8 (Accession number: VKPMB-S926), Rhodococcus rhodochrous M33, Rhodococus pyridinovorans, and Escherichia coli MT-10822 (Accession number: FERM BP-5785).

34. A biocatalyst composition obtainable by the method according to claim 21.

35. Use of the biocatalyst composition according to claim 34 for preparing an aqueous solution of (meth-) acrylamide in a process of converting (meth-) acrylonitrile to (meth-) acrylamide.

36. A method of producing an aqueous (meth-) acrylamide solution from (meth-) acrylonitrile comprising the steps of:
(a) providing an aqueous suspension comprising a biocatalyst which is capable of converting acrylonitrile to acrylamide, which aqueous suspension is an aqueous fermentation broth;
(b) sequentially in either order or simultaneously
(b1) concentrating the aqueous suspension comprising the biocatalyst to a concentration of at least 3% (w/w); and
(b2) reducing the temperature of the aqueous suspension comprising the biocatalyst, to a temperature of below 8° C.,
thereby forming a concentrated aqueous suspension; and
(c) maintaining the concentrated aqueous suspension of step (b) at a temperature of below 8° C.;
(d) providing a ready to use aqueous composition from the aqueous suspension of step (c) by
(i) increasing the temperature of the concentrated aqueous biocatalyst composition to a temperature of at least 8° C., preferably at least 15° C.; and
(ii) diluting the concentration of the aqueous biocatalyst composition to a concentration of below 3% (w/w);
in which (i) and (ii) are conducted simultaneously or sequentially in either order;
(e) contacting (meth) acrylonitrile in an aqueous medium with the ready to use aqueous biocatalyst composition; and
(f) conducting the conversion reaction of (meth-) acrylonitrile to produce the aqueous (meth-) acrylamide solution.

37. A method according to claim 36 comprising one or more of the features of claim 22.

38. An aqueous (meth-) acrylamide solution obtainable by claim 36.

39. A method for producing a polyacrylamide comprising the steps of:
(a) providing an aqueous suspension comprising a biocatalyst which is capable of converting acrylonitrile to acrylamide, which aqueous suspension is an aqueous fermentation broth;
(b) (b) sequentially in either order or simultaneously
(b1) concentrating the aqueous suspension comprising the biocatalyst to a concentration of at least 3% (w/w); and
(b2) reducing the temperature of the aqueous suspension comprising the biocatalyst to a temperature of below 8° C.,
thereby forming a concentrated aqueous suspension;
(c) maintaining the concentrated aqueous suspension of step (b) at a temperature of below 8° C.;
(d) providing a ready to use aqueous biocatalyst composition from the aqueous suspension of step (c) by
(i) increasing the temperature of the concentrated aqueous biocatalyst composition to a temperature of at least 8° C., preferably at least 15° C.; and
(ii) diluting the concentration of the aqueous biocatalyst composition to a concentration of below 3% (w/w);
in which (i) and (ii) are conducted simultaneously or sequentially in either order;
(e) contacting (meth) acrylonitrile in an aqueous medium with the ready to use aqueous biocatalyst composition;
(f) conducting the conversion reaction of (meth-) acrylonitrile to produce the aqueous (meth-) acrylamide solution; and
(g) polymerising the aqueous (meth-) acrylamide solution obtained in step (g) to polyacrylamide.

40. A method according to claim 39 which incorporates one or more of the features of claim 22.


SEQUENCE LISTING (4 sequences)

SEQ ID NO: 1 - DNA, 612 bp, Rhodococcus rhodochrous
gtgagcgagcacgtcaataagtacacggagtacgaggcacgtaccaaggcgatcgaaaccttgctgtacgagcgagggctcatcacgcccgccgcggtcgaccgagtcgtttcgtactacgagaacgagatcggcccgatgggcggtgccaaggtcgtggccaagtcctgggtggaccctgagtaccgcaagtggctcgaagaggacgcgacggccgcgatggcgtcattgggctatgccggtgagcaggcacaccaaatttcggcggtcttcaacgactcccaaacgcatcacgtggtggtgtgcactctgtgttcgtgctatccgtggccggtgcttggtctcccgcccgcctggtacaagagcatggagtaccggtcccgagtggtagcggaccctcgtggagtgctcaagcgcgatttcggtttcgacatccccgatgaggtggaggtcagggtttgggacagcagctccgaaatccgctacatcgtcatcccggaacggccggccggcaccgacggttggtccgaggaggagctgacgaagctggtgagccgggactcgatgatcggtgtcagtaatgcgctcacaccgcaggaagtgatcgtatga

SEQ ID NO: 2 - protein, 203 aa, Rhodococcus rhodochrous
VSEHVNKYTEYEARTKAIETLLYERGLITPAAVDRVVSYYENEIGPMGGAKVVAKSWVDPEYRKWLEEDATAAMASLGYAGEQAHQISAVFNDSQTHHVVVCTLCSCYPWPVLGLPPAWYKSMEYRSRVVADPRGVLKRDFGFDIPDEVEVRVWDSSSEIRYIVIPERPAGTDGWSEEELTKLVSRDSMIGVSNALTPQEVIV

SEQ ID NO: 3 - DNA, 690 bp, Rhodococcus rhodochrous
atggatggtatccacgacacaggcggcatgaccggatacggaccggtcccctatcagaaggacgagcccttcttccactacgagtgggagggtcggaccctgtcaattctgacttggatgcatctcaagggcatatcgtggtgggacaagtcgcggttcttccgggagtcgatggggaacgaaaactacgtcaacgagattcgcaactcgtactacacccactggctgagtgcggcagaacgtatcctcgtcgccgacaagatcatcaccgaagaagagcgaaagcaccgtgtgcaagagatccttgagggtcggtacacggacaggaagccgtcgcggaagttcgatccggcccagatcgagaaggcgatcgaacggcttcacgagccccactccctagcgcttccaggagcggagccgagtttctctctcggtgacaagatcaaagtgaagagtatgaacccgctgggacacacacggtgcccgaaatatgtgcggaacaagatcggggaaatcgtcgcctaccacggctgccagatctatcccgagagcagctccgccggcctcggcgacgatcctcgcccgctctacacggtcgcgttttccgcccaggaactgtggggcgacgacggaaacgggaaagacgtagtgtgcgtcgatctctgggaaccgtacctgatctctgcgtga

SEQ ID NO: 4 - protein, 229 aa, Rhodococcus rhodochrous
MDGIHDTGGMTGYGPVPYQKDEPFFHYEWEGRTLSILTWMHLKGISWWDKSRFFRESMGNENYVNEIRNSYYTHWLSAAERILVADKIITEEERKHRVQEILEGRYTDRKPSRKFDPAQIEKAIERLHEPHSLALPGAEPSFSLGDKIKVKSMNPLGHTRCPKYVRNKIGEIVAYHGCQIYPESSSAGLGDDPRPLYTVAFSAQELWGDDGNGKDVVCVDLWEPYLISA